(19) United States
(12) Patent Application Publication
(10) Pub. No.: US 2009/0129974 A1
(43) Pub. Date: May 21, 2009

(54) Air quality enhancing ceiling paddle fan

(76) Inventor: John J. McEllen, Chagrin Falls, OH (US)

Correspondence Address: L. Lawton Rogers, III, 3260 Luvan Boulevard, Georgetown, SC, 29440, US

(21) Appl. No.: 11/888,394

(22) Filed: Aug. 2, 2007

(51) Int. Cl. A61L 9/20; F04D 29/00

(52) U.S. Cl. 422/24; 422/121; 422/108; 416/5; 416/146 R; 416/244 R; 416/241 R

(57) ABSTRACT

A ceiling paddle fan fixture for improving the quality of the air in the room and various methods of enhancing room air quality, with particular application in public spaces such as hospitals, health care institutions, dormitories, schools and offices. A UV-C source and an air mover are combined in a single fixture which uses a low intensity UV-C source and a paddle ceiling fan to significantly increase the mixing of the treated with the untreated room air. Sterilization of room air is achieved by the passage of a high volume of air at a relatively slow speed through a relatively low intensity UV-C field.

### BACKGROUND OF THE INVENTION

The present invention relates to a ceiling paddle fan fixture for improving the quality of the air in the room and to various methods of enhancing room air quality. The present invention finds particular application in public spaces such as hospitals, health care institutions, dormitories, schools and offices.

Ceiling paddle fans are quiet, efficient and high volume air mixers and are well known and often used both indoors and outdoors. The large slowly rotating blades are designed to move large volumes of air at a relatively slow speed, creating a cooling effect by causing more rapid evaporation of perspiration. Indoors, they are often used as a less expensive alternative (or supplement) to air conditioning. Outdoors, they are often used on porches, verandas and patios to supplement natural breezes.

The blades of paddle fans are typically slanted at an angle of about 10° to 15° from the horizontal on the leading edge—i.e., when operated in a clockwise direction, the blades will cause a generally horizontal and downward flow of air. This causes evaporation and a general sensation of cooling. Conversely, if the blades are operated in a counterclockwise direction, air will tend to be pulled up vertically and then redistributed in a downward direction. If used in this manner, the fans may help to redirect heated air back into the lower room which would be useful in colder months. In addition, operation in a way that pulls air upward will move recently generated germs from proximity of those generating them immediately through the UV-C field and thus tend to effect sterilization before the contaminated air is circulated to others in the room.

Paddle fans can typically be operated at a range of revolutions (typically three speeds) which enables the user to control drafts. Because of their potential to circulate air and reduce energy consumption, paddle fans have been widely adopted.

In recent years, a much broader selection of ceiling fans has become available with less emphasis on function and price and more emphasis on design and aesthetic considerations. “Light kits” are often added to ceiling paddle fans to increase utility and energy-efficient models feature angled paddle blades and two-way fluorescent lighting (up, down or both). Some state governments are now specifying energy standards for paddle ceiling fans—e.g., they may use only fluorescent sockets, energy efficient motors, etc.

As disclosed in applicant's co-pending application Ser. No. 11/823,507 filed Jun. 28, 2007, the entire disclosure of which is hereby incorporated by reference herein, the use of short wave ultra violet (“UV-C”) sources for the sterilization of air is well known. The usefulness of UV-C irradiation on air quality lies in the effect on germs (microorganisms) transmitted in aerosolized form. Such infectious germs are generally less than 0.3 microns in diameter and are suspended or “float” in the air.

Different types of microorganisms vary significantly in their resistance to UV-C irradiation. For example, spores such as anthrax have a “cell wall” (like bacteria) as well as an outer “shell” which must be penetrated by the UV-C energy. Viruses such as influenza, the common cold, SARS, measles and small pox do not have a cell wall and are about five times more susceptible to UV-C radiation than spores. Bacteria with a cell wall such as tuberculosis, even extended drug resistant (XDR) TB, may be ten times more vulnerable to UV-C radiation than anthrax spores. The UV-C “dose” needed to destroy germs is generally expressed as joules (one UV-C watt of energy for one second) per square meter; or the equivalent γj/cm²—micro-joules per square centimeter.

It is desirable to effect air sterilization within the room where the germs originate. However, there are safety issues. Keratoconjunctivitis (external inflammation of the eye) and erythmea (reddening of the skin) can result from overexposure to UV-C and the National Institutes for Occupational Safety and Health (NIOSH) recommends an upper limit on the amount of UV-C radiation for the safety of personnel in the room, i.e., 6 m j/cm²—6 micro-joules per square centimeter over a continuous eight-hour period. Although they may be modified from time to time, the NIOSH guidelines must be considered in the design of fixtures for public spaces and home use.

Because of safety considerations, air sterilization products (e.g., in-duct, ceiling and floor mounted fixtures) generally avoid UV-C radiation into a room and have attempted to confine UV-C radiation to the interior of a closed (i.e., UV-C baffled) chamber, and pass air through the baffled chamber for sterilization.

Initial efforts to effect air sterilization with a UV-C field external of the fixture transmitted an intense UV-C beam at a room height well above the “eye level” of people occupying the room, i.e., generally considered to be approximately 60 inches above the floor. Germ reduction occurred in the air passing through the beam as a result of convection currents and ventilation systems. While the intensity of the beam was effective in sterilizing the air passing through the beam, the volume and velocity of the air passing through the beam was not controlled and, being thus subject to external forces, such devices have generally been ineffective. In addition, the narrowing of the beam vertically, typically through the use of louvers, wasted most of the UV-C energy making such fixtures highly inefficient.

Experiments have been conducted with high intensity UV-C sources in which the amount of room air passing through the field of UV-C radiation is increased by the use of independent air movers such as floor fans. Such air movers serve to stir or mix the air in the room, combining treated air with untreated air, so that mixed air is circulated back through the UV-C killing field. Experimental data suggest that such systems are much more effective in reducing the concentration of microorganisms in the room than conventional “beam” sterilizers.

It has been proposed to combine the air sterilization and air movement features needed to effectively sterilize air. One such proposal was for a ceiling mounted, squirrel cage motor driven, impeller with a high intensity UV-C array located circumferentially around the impeller so that air drawn upwardly into the impeller could be laterally exhausted by the UV-C array into the room. Such device proved impractical because of its large size, interference with air flow by the motor and the baffling required to avoid an unacceptable radiation level in the lower part of the room.

More recently, applicant's co-pending application discloses an improved ceiling device of much smaller size that uses a low intensity source with easily replaced arcuate UV-C bulbs. Such devices rely on a relatively concentrated centralized air intake and the mixing of treated with untreated air by the exhaustion of treated air along the ceiling and are particularly suitable for rooms with relatively low ceilings.

This application is directed to the combination of a UV-C source and an air mover in a single fixture which uses a low intensity UV-C source and a paddle ceiling fan to significantly increase the mixing of the treated with the untreated room air. Sterilization of room air is achieved by the passage of a high volume of air at a relatively slow speed through a relatively low intensity UV-C field. Thus, the novel paddle ceiling fan fixture of the present invention provides room lighting, air circulation and safe and effective room air sterilization.

In one aspect, this invention relates to a ceiling fan fixture that utilizes movable sources of radiation in providing effective room air sterilization.

In another aspect, this invention relates to a ceiling fan fixture having a diffused source of radiation for effective room air sterilization.

In still another aspect, this invention relates to the establishment of a UV-C radiation field by directing radiation across the upper surface of the paddle blades.

In yet another aspect, the intensity of the UV-C radiation may be varied independently of, or as a function of, air flow to match the amount of room air mixing. The UV-C source may also be automatically varied as a function of the presence of persons or objects in an area of the room above eye level.

In a further aspect, the fixture of the present invention is fully integrated with smoke and carbon monoxide detection to provide a holistic safety approach for germ killing, alarms and emergency back up lighting.

In yet a further aspect, the fixture of the present invention may originate the UV-C field from within the canopy from which the paddle fan is suspended.

The present invention aims to enable common paddle-type ceiling fans to incorporate UV-C air sterilization as a countermeasure to deadly aerosolized infectious diseases like influenza, tuberculosis, and SARS, as well as biological weapons such as anthrax and small pox. By combining UV-C radiation with ceiling paddle fans, it will be possible to equip schools, government buildings, military barracks, dormitories, assisted living centers, hospitals and health care facilities with sufficient air sterilization potential to address a broad range of emergencies and health crises.

Many other objects and advantages will be apparent from the following detailed description of preferred embodiments when read in conjunction with the appended drawings.

### THE DRAWINGS

FIG. 1 is a pictorial representation of one embodiment of the fixture of the present invention.

FIG. 2 is a pictorial representation in partial section of the fixture of FIG. 1 with selected components omitted in the interest of clarity.

FIG. 3 is a pictorial representation of a horizontal section through the fixture above the fan blades illustrating an alternative arrangement of UV-C and visual light sources in the illumination unit.

FIG. 4 is a pictorial representation in partial section showing an embodiment wherein the UV-C source of radiation is located in the canopy from which the paddle fan is suspended.

FIG. 5 is a pictorial representation in cross-section of one embodiment of a fan blade having a shape that provides shielding against downward emanation of UV-C radiation.

### THE DETAILED DESCRIPTION

As shown in FIG. 1, one embodiment of the fixture of the present invention includes a ceiling mount 10 through which electric power is conventionally supplied through a hollow rod or pipe 12 which serves as an electrical conduit to, and support for, the suspended fan assembly 14. The ceiling mount 10 may take the form of a flat round medallion or a 2′×2′ or 2′×4′ panel for integration in and coordination with common acoustic ceilings. The mount 10 may be UV-C absorptive to avoid reflection of UV-C radiation into the lower part of the room and discoloration of ceiling materials.

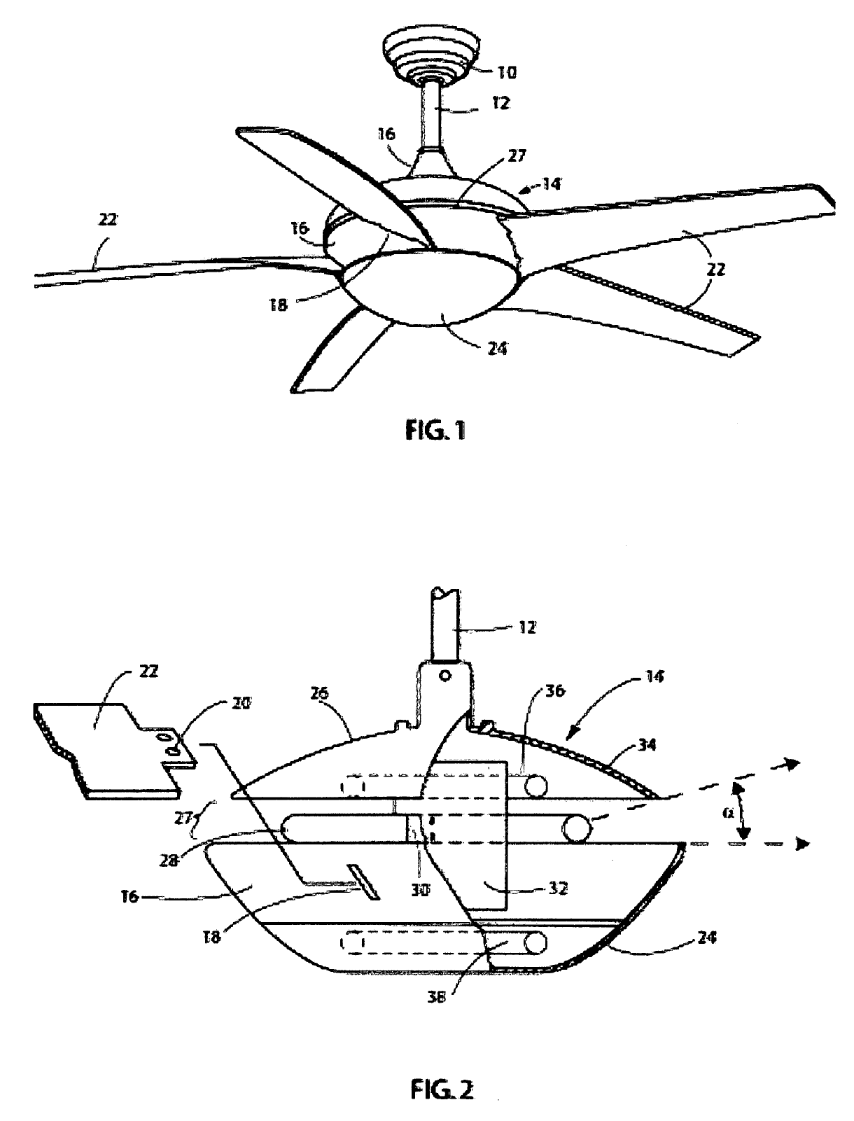

In the embodiment of FIGS. 1 and 2, the fan assembly 14 includes a rotor assembly 16 having a plurality of slots 18 spaced around the periphery thereof to receive the bayonet ends 20 of paddle fan blades 22. Below the rotor assembly 16 is an illumination unit 24 for providing room lighting. Above the rotor unit 16 is an illumination unit 26 for “up-lighting” or proving indirect room lighting by illuminating the ceiling from which the fan assembly 14 is suspended. As shown more clearly in FIG. 2, there is an opening 27 between the rotor unit 16 and the upper room illumination unit 26. In the embodiment of FIGS. 1 and 2, it is the opening 27 through which the UV-C radiation passes from a source internal of the fan assembly 14 to establish the radiation field in the upper part of the room.

With reference to FIG. 2, the source of UV-C radiation is desirably a pair of semi-circular lamps 28 mechanically supported by the upper illumination assembly 26 by suitable conventional brackets (not shown) and operably connected at the ends to a pair of sockets 30 in turn connected through a ballast and control unit (not shown) carried by the upper illumination assembly 26. The circular arrangement of the UV-C emitting lamps 28 provides space for the motor 32 that drives the rotor unit 16 and the paddle fan blades 22 attached thereto. It also provides space for the various electrical and electronic components that control the operation of the fan assembly 14, including a conventional receiver for remote control operation.

The UV-C lamps 28 are desirably located along the upper edge of the rotor unit 16 so that the rotor unit defines the lower edge of the opening 27 and prevents the downward emission of direct UV-C radiation into the lower part of the room. The upper end of the opening 27 is defined by the UV-C absorbing cover 34 of the upper illumination assembly 26. The angle α through which direct UV-C radiation may exit the opening 27 is between about 10 and about 45 degrees. The upward limitation of the angle α limits the indirect radiation of the lower part of the room by reflection of direct radiation by the ceiling.

So that visible room light is controlled solely as a function of the control of the source 32, the source 28 of UV-C radiation may be provided with a filter for visible light emanating from the UV-C source. “Notched” dichroic or thin film filters are contemplated for this application, but other suitable conventional filtering may be employed. In this way, hospital patients, e.g., may have the benefits of continuous air sterilization at night without potential sleep impairment from the visual light components of the UV-C source.

The upper illumination unit 26 may also include a circular fluorescent bulb 36 the visible light from which passes through a glare reducing or diffusing glass cover 34 opaque to UV-C radiation.

The lower illumination unit 24 may also house a circular fluorescent bulb 38 with a glare reducing or diffusing cover to illuminate the lower part of the room.

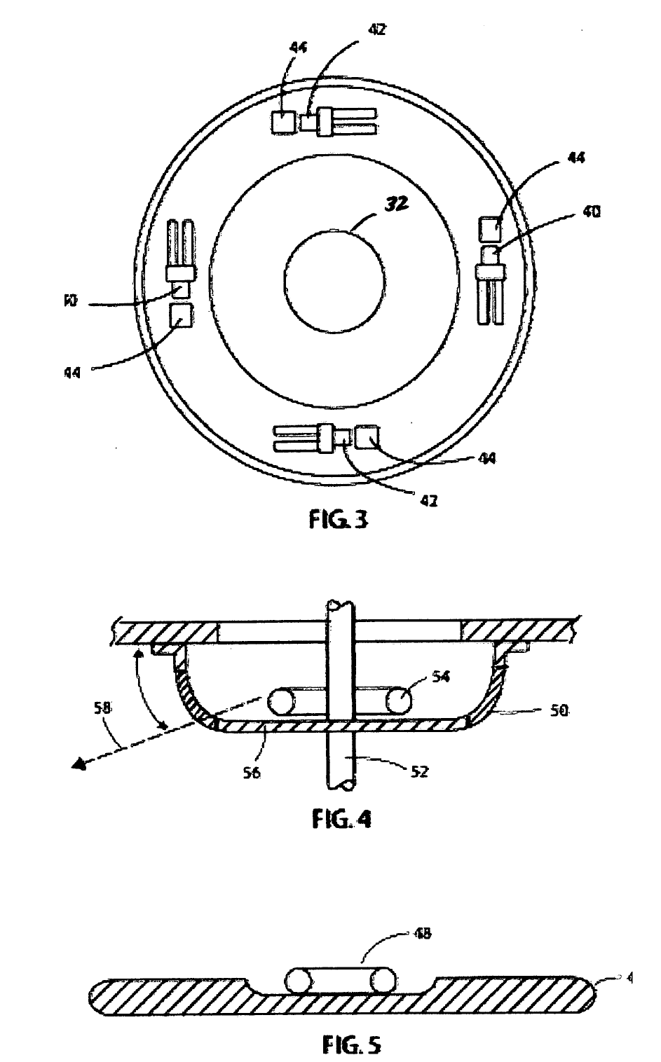

The UV-C source 28 may be any suitable conventional type such as light emitting diodes (LED), high intensity discharge (HID) or fluorescence, and include any required starters, ballasts or other current regulators. They may, for example, may be in the shape of conventional two armed PL lamps and the sources of visible illumination may be located on the same plane as shown in FIG. 3.

With reference to FIG. 3, the illumination unit 26 may contain alternating UV-C sources 40 and visible light sources 42, each provided with an appropriate ballast 44.

The sources 36 of UV-C radiation may be any of suitable conventional type including fluorescents, cold cathode fluorescent, HID or LED sources and may include any required starters, ballasts or other current regulators. The electrical connection to all of the sources may be automatically interrupted by the mechanical removal of a blade 22 from the motor unit 16 and may be independently switched so that the sources may be operated independently of the rotation of the blades 22.

The blades or paddles 18 may be any suitable conventional configuration designed to move air and to provide appropriate aesthetics

The control circuit (not shown) is desirably located in the housing proximate to the motor 32 and is desirably remotely controlled in a conventional manner. The fixture may also include electrical ports for the addition of smoke, carbon monoxide, or motion detectors, and the UV-C radiation, visible light and blade rotation may be independently controlled as a function of these sensors or the desires of those in the room.

The capability of the UV-C ballast and radiation source to be adjusted provides a larger or smaller amount of UV-C energy in the radiation field. Such adjustment can be made via manual or remotely controlled switches, or may be triggered by sensors and/or detectors of microorganisms and/or concentration levels of microorganisms in the room. For instance, the presence of anthrax spores may be detected via imaging technologies triggering an immediate and sustained pulse of voltage resulting in UV-C irradiation capable of eliminating a pre-determined amount of anthrax spores within the space. Since anthrax spores would require other activity (evacuation), the limits of the predetermined eye level threshold could be increased due to the short duration of human exposure.

It is desirable that the unit contain indicator lights, e.g. LEDs, or glass surfaces, to indicate to those in the room that the UV-C source is in operation, This is especially true when the visible light from the UV-C source is suppressed and cannot provide such indication.

A manual or automatic override may also be provided to allow a user to increase the UV-C intensity for contingencies such as flu, bird flu, outbreaks/epidemics; bio-terrorist attack; black mold, etc. When the intensity of UV-C radiation is temporarily increased, it is desirable to provide a further indicator lights to those in the room, e.g. “excess UV-C is in operation”-“may exceed safe threshold-protect eyes and skin.”

In another embodiment, the UV-C radiation field may be established by sources in the ceiling medallion or canopy from which the fan is suspended. As shown in FIG. 4, the canopy 50 from which the fan (not shown) may be conventionally suspended by a rod 52 may contain one or more UV-C sources 54 as earlier described. The lower portion 56 of the canopy 50 may be opaque to UV-C radiation and thus serve to limit that angle at which radiation 58 enters the room.

By limiting the downward angle, direct radiation may not reach eye level within the dimensions of the room and the radiation will be reduced in intensity over the distance traveled. In addition, the radiation will pass through the space through which the fan blades rotate so that the radiation will be periodically interrupted by the rotating blades. This will reduce the time that direct radiate is present at any specific spot in the room below eye level and permit a higher radiation intensity than would otherwise be acceptable.

The control circuit as described supra may be housed within the canopy 50 or within the fan housing as desired.

In another embodiment, the source of UV-C radiation may be carried by one or more of the paddle blades. As shown in FIG. 4, the blades 46 should be of a material and be configured and have a width to effectively baffle or shield the lower part of the room from radiation in excess of that desired. The upper surface of the blades 18 may be covered with any suitable UV-C radiation absorbing material, e.g., titanium oxide paint, to aid in the shielding. The UV-C source 48 may be any suitable conventional source including a longitudinal tube disposed in a longitudinal groove in the upper surface of the blade 46, or may alternatively be a series of discrete sources such as LEDs or PL lamps. Diffusion of the radiation is achieved by rotation through space.

Microorganisms are killed in the air through which the blades move and in the air circulated through the upward and horizontally radiation field extending from each source as it is moved. Since the sterilized air is constantly being mixed with air that has not been sterilized by the fan blades, a significant reduction in viable airborne microorganisms in the air in the lower part of the room where people are present is achieved.

The fixture may include other air improvement features such as filters to remove dust from the air circulated through the fixture. For example, the lower surface of the blades 46 may be provided with removable conventional “stick-on” filters where they will not interfere with UV-C radiation absorption and the aesthetics of the fixture. Alternatively, the upper part of the blades may include photocatalytic crystal films or other coatings which reduce fluorocarbons (smoke, odors, etc.), dust or both.

Power to the UV-C sources 48 on the blades 46 may be conventionally provided by a commutator ring and in such application it may be desirable that the control circuit interrupt power when the blades are not turning or when a blade is removed from the fan.

As understood from the embodiment of FIGS. 1 and 2 supra, UV-C radiation may be emitted from or through slits in the fan housing upwardly, horizontally and at a slight downward angle toward the blades 46. Where the blades have an UV-C reflective upper surface, radiation will be reflected upwardly from the blades as they rotate creating movable sources and enhancing diffusion of the radiation field.

By limiting the downward angle, direct radiation may not reach eye level within the dimensions of the room and will be reduced in intensity over the distance traveled. In addition, the interruption of the radiation by the rotating blades will reduce the time that direct radiation is present at any specific spot in the room below eye level and permit a higher radiation intensity than would otherwise be acceptable.

While the foregoing is a description of preferred embodiments, many variations and modifications will naturally occur to those of skill in this art from a perusal hereof. The invention is therefore not to be limited to the embodiments disclosed, but defined only by the claims when accorded a full range of equivalents.

What is claimed is:

1. An in-room air sterilizer for a room having a ceiling sufficiently high to safely suspend a paddle ceiling fan therefrom, said sterilizer being adapted for connection through the ceiling to a source of electrical energy, said air sterilizer comprising:
a fan housing;
a hollow rod connected to said fan housing for suspending said fan housing from the ceiling and for providing power to said fan housing;
a decorative medallion surrounding said rod adapted to be located adjacent the ceiling,
said fan housing including:
a rotor assembly with plural laterally extending fan blades,
a motor positioned at about the height of said rotor for driving said rotor,
a source of UV-C radiation positioned above said rotor assembly for providing a circumferential UV-C radiation field that does not exceed a predetermined radiation intensity threshold at a predetermined height above the floor, and
a source of visual light positioned below said rotor assembly for providing room illumination,
so that rotation of said rotor assembly to rotate said fan blades effects the mixing of air within the room and the movement of air through the radiation field.

2. The sterilizer of claim 1 where the radiation field is both internal and external of said housing.

3. The sterilizer of claim 1 wherein said source of UV-C radiation includes a source of visual light for selectively illuminating the ceiling.

4. The sterilizer of claim 3 where said source and visual light is separate from said source of UV-C.

5. The sterilizer of claim 1 wherein said UV-C source includes a filter of visible light.

6. The sterilizer of claim 5 wherein said filter is a notched dichroic filter.

7. An in-room air sterilizer adapted for connection to a source of electrical energy, said air sterilizer comprising:
an electric motor driven paddle ceiling fan; and
a source of UV-C radiation carried by said ceiling fan for establishing a radiation field within the room air proximate to said fan that does not exceed a predetermined radiation intensity threshold at a predetermined height above the floor.

8. The sterilizer of claim 7 where said source is stationary and radially inward of the paddles of said ceiling fan.

9. The sterilizer of claim 7 where said source rotates with the paddles of said ceiling fan.

10. The sterilizer of claim 7 where said source is selectively adjustable

11. The sterilizer of claim 10 where said source is electronically adjustable.

12. The sterilizer of claim 7 including a detector of microorganisms where said source is adjusted responsibly to said detector.

13. A ceiling paddle fan including a stationary source of visible light and a movable source of invisible radiation

14. The fan of claim 13 wherein said stationary source is one or more of fluorescent, incandescent, LED or HID.

14. The fan of claim 13 wherein said invisible radiation is UV-C.

16. A method of sterilizing room air comprising the steps of:
(a) providing a ceiling mounted paddle fan with a source of UV-V radiation;
(b) using the UV-C source to creating an UV-C radiation field above and circumferentially around the fan;
(c) using the paddle fan to move air through the radiation field.

17. In room with infected persons and uninfected persons not in immediate proximity to the infected persons, a method of reducing the risk of infecting the uninfected persons from germs introduced into the air by the infected persons by moving the air immediately proximate to the infected persons upwardly into a UV-C radiation field in the upper part of the room for subsequent mixing with room air prior to circulation to the immediate proximity to the uninfected persons.

18. A method of improving air quality by using a paddle fan to circulate air through a circular field of UV-C radiation.

19. A canopy for a ceiling paddle fan comprising:
a housing adapted for mounting to a ceiling around the rod by which a paddle fan is suspended; and
a source of UV-C radiation carried within said housing for providing a circumferential UV-C radiation field around said canopy,
said housing being sufficiently opaque to UV-C radiation so that the radiation does not exceed a predetermined radiation intensity threshold at a predetermined height above the floor of the room.

20. A paddle blade for a ceiling mounted fan comprising:
an elongated generally flat blade member suitable for moving air when rotated in a generally horizontal plane by a ceiling fan; and
a source of UV-C radiation carried by said member in position to establish a radiation field above said member as said member is rotated in a generally horizontal plane.

21. An in-room air sterilizer comprising:
an electric motor driven paddle ceiling fan;
a source of UV-C radiation carried by said ceiling fan for establishing a radiation field within the room air proximate to said fan;
a parameter detector; and
electronic controls that automatically modify the intensity of the UV-C radiation in response to the parameter detected.

22. The sterilizer of claim 21 where parameter is germ population

23. The sterilizer of claim 21 where parameter is type of germ (e.g. spores)

24. The sterilizer of claim 21 where parameter is motion within the room

25. The sterilizer of claim 21 where the parameter is the volume of air moved by the fan

26. The sterilizer of claim 21 where UV-C intensity is a predetermined minimum.

27. The sterilizer of claim 21 where UV-C source is always “on” to avoid response delay.

28. An in-room air sterilizer comprising:
an electric motor driven ceiling paddle fan;
a source of UV-C radiation carried by said paddle fan for establishing a radiation field within the room air proximate to said fan; and
electronic controls for modifying UV-C radiation intensity independently of volume of air moved.

29. The sterilizer of claim 28 wherein said field is sufficiently strong to kill common viruses only laterally and upwardly of said sterilizer.

30. An in-room air sterilizer adapted for connection to a source of electrical energy, said air sterilizer comprising:
an electric motor driven impeller for moving room air vertically within the room; and
a source of UV-C radiation carried by said impeller for radiating the room air in proximity to said impeller as it rotates.

31. The sterilizer of claim 30 wherein said impeller is a plural paddle fan.

32. A method of sterilizing room air comprising the step of passing an air moving surface through the room air while emitting UV-C radiation from the passed surface.

33. An in-room air sterilizer for a room having a ceiling sufficiently high to safely suspend a paddle ceiling fan therefrom, said sterilizer being adapted for connection through the ceiling to a source of electrical energy, said air sterilizer comprising:
a fan housing;
a hollow rod connected to said fan housing for suspending said fan housing from the ceiling and for providing power to said fan housing;
said fan housing including:
a rotor assembly with plural laterally extending fan blades,
a motor for driving said rotor assembly, and
a source of UV-C radiation carried by at least one of said blades for irradiating room air proximate thereto that does not exceed a predetermined radiation intensity threshold at a predetermined height above the floor.

34. A method of sterilizing the air in a room by:
(a) providing a plurality of room air engaging surfaces at least one of which includes a source of UV-C radiation; and
(b) rotating the surfaces in a generally horizontal plane to thereby simultaneously move air and create a rotating field of UV-C radiation.

35. A method of improving room air quality comprising the step of moving a source of UV-C radiation through the air in the room.

36. The method of claim 35 wherein the movement is rotation about a vertical axis

37. The Method of claim 35 wherein the movement is in a horizontal plane

38. A method of improving room air quality comprising the step of circulating air by a fan blade carrying an UV-C source.

39. A ceiling fixture with a paddle fan, a stationary source of visible magnetic radiation, and a movable source of invisible magnetic radiation.

40. A method of creating a radiation field for air sterilization comprising the steps of:
(a) creating a radiation field extending laterally from a central source; and
(b) moving a reflecting surface through the field in a generally horizontal plane to reflect some of the energy upwardly.

41. An room air sterilizer comprising:
a ceiling paddle fan;
a source of UV-C radiation carried by said fan in position to radiate the supper surface of the paddles,
the paddles of said fan having a UV-C reflective upper surface to reflect UV-C radiation upwardly toward the ceiling.
